(19) United States
(12) Patent Application Publication
(10) Pub. No.: US 2004/0126877 A1
(43) Pub. Date: Jul. 1, 2004

(54) Repressors for hiv transcription and methods thereof

(76) Inventors: Man-Wook Hur, Gyoungki-do (KR); Deug-Lim Chong, Seoul (KR)

Correspondence Address: Pennie & Edmonds, 1155 Avenue of the Americas, New York, NY, 10036-2711, US

(21) Appl. No.: 10/475,681

(22) PCT Filed: Apr. 19, 2002
(86) PCT No.: PCT/KR02/00730

(30) Foreign Application Priority Data
Apr. 20, 2001 (KR) 2001/21449
Apr. 18, 2002 (KR) 2002/21307

(51) Int. Cl. C12Q 1/70; C12Q 1/68; C07H 21/04

(52) U.S. Cl. 435/320.1; 435/6; 536/23.72; 530/350; 435/5

(57) ABSTRACT

The present invention relates to repressors suppressing production of acquired immune deficiency syndrom (AIDS) viral genome (RNA) and methods repressing transcription thereof. The invention, more specifically, relates to fusion proteins repressing AIDS viral genomes (RNA) of a protein selected from group consisting of proteins strongly repressing activities of proteins such as Sp1, NF-kB, proteins repressing transcriptional activities by strongly condensing chromatin, and proteins which are able to bind around AIDS viral promoter; and protein (for example Tat or Tat derivatives) recognizing short RNA strand (TAR) and methods repressing transcription using same. The said fusion proteins dramatically show effect repressing production of HIV-1 genome (RNA) by delivering repressing proteins like Sp1, NF-k B to HIV-1 LTR using protein recognizing short RNA strand as a carrier. The invention shows transcription inhibitory effect of HIV by targeting transcription-repressing fusion proteins to HIV LTR.

### BACKGROUNG OF THE INVENTION

1. Field of the Invention

The present invention relates to the repressor for repressing production of acquired immune deficiency syndrome (AIDS) viral genomes (RNA) from proviral or nuclear long terminal repeat (LTR) promoters. More specifically, the present invention relates to fusion proteins for repressing transcription of AIDS viral genomes (RNA), comprising polypeptide sequence selected from the group consisting of: proteins strongly repressing activities of proteins such as Sp1 or NF-κB; proteins repressing transcription by strongly condensing chromatin; protein such as zinc fingers able to bind on AIDS viral promoters; and polypeptide sequence (e.g. Tat protein or Tat derivatives) recognizing short RNA strands (HIV short transcript).

The fusion proteins have remarkable effects to repress production of the human immunodeficiency virus (HIV)-1 RNA, when proteins strongly repressing activities of proteins such as Sp1 or NF-κB and proteins repressing transcription by strongly condensing chromatin are targeted around the expression control region of HIV-1 LTR promoters by using short RNA strand recognizing proteins (e.g. Tat proteins and mutants thereof) as missile.

2. Description of the Prior Art

In general, HIV entry is known to require an binding of the viral envelope protein(GP120) and cell membrane surface receptors. Following binding, the virus fuses with the cell and injects their viral materials. After fusion, viral genome(RNA) is changed to DNA by reverse transcriptase. Additionally, by integrase DNA type viral genome is then integrated into DNA in the host cell where it exists for the life of the cell as a “provinis.” The proviral HIV genome produces the genomic RNA using an expression system of host cell (transcription process), and mass-produces components (proteins, capsids) necessary for proliferation in cytoplasm (translation and protein cleavage, protease function) by using the viral genome. After an assembly process, the HIV genome is then released from the host cell and proliferates. If it is beyond a critical point, 10 billions of new HIVs are proliferated a day on the average. As a result, after infection AIDS is incurable.

Many anti-AIDS drugs have been developed as antibodies (vaccines) or compounds for inhibiting the above-described processes. Anti-AIDS drugs widely used are inhibitors for reverse transcription process and protease. Although vaccines for inhibiting a process of recognizing host cells has been developed, they have difficulty in suppressing HIV due to rapid mutations of the viral envelope protein. In addition, HIV reverse transcriptase inhibitors and protease inhibitors act as general. HIV suppressors, and various reverse transcriptase inhibitors or protease inhibitors have been developed and marketed in many drug companies. They have effect on inhibition of growth of HIV in the early stage but have some problem such as toxicity and rapid resistance.

Furthermore, the mechanism study on HIV injection into host cell genomes or therapeutic agent on the process has not been developed. Anti-sense gene therapy and ribozyme gene therapy have been developed since 1997 due to the increasing interest on gene therapy to treat AIDS. A first phase clinical experiment have been in progress since 1997, but the therapy have a deep-seated problem in obtaining viral resistance.

Many studies on expression control mechanism of growth of viral RNA have demonstrated that transcription control protein in specific host cell is important for RNA expression. Recently, the study have demonstrated that Tat acting with TAK (Tat-associated kinase or PTEF) has great effect on activity of RNA synthetic enzyme. As a result, the recent study places the focus on inhibitors (e.g. Tat analogue protein fragments, TAR analogue RNA segments, TAK inhibitors) for production of RNA by repressing actions of Tat or TAK.

The above-mentioned therapeutic agents extend life to a certain degree by slowing the process of disease but lose their effect due to rapid resistant viral production. Those therapeutic agents have the basic problem of expression possibility that viral DNA genomes (provirus) existing in the host cell may be expressed into viral RNA because proviral genomes can exist regardless of the above-described agents, vaccines and gene therapy.

Accordingly, the best way to overcome the problems of the existing agents is to suppress a transcription process that proviral LTR is expressed into genomic RNA. The present invention provides fusion proteins for repressing HIV transcription regulating the expression of AIDS viral RNA. In other words, the fusion proteins of the present invention prevent viral DNA genomes in host cell from being expressed into viral RNA genomes, and then block production of components (RNA genomes, proteins, capsids) necessary for viral proliferation, thereby basically inhibiting proliferation of virus and production of resistant virus. Therefore, the present invention overcomes the problems of the conventional agents and provides an innovating AIDS therapeutic agent.

### SUMMARY OF THE INVENTION

In accordance with the present invention, an object of the present invention is to provide a new biological therapeutic agent for basically repressing viral genome(RNA) necessary for proliferation of AIDS and overcoming resistance of virus and a method of repressing HIV transcription to treat AIDS.

In order to accomplish the above-mentioned object, the present invention provides a fusion protein for repressing HIV transcription, comprising: a polypeptide or compound selected from the group consisting of a) strongly repressing activity of transcription factors such as Sp1 or NF-_{K}B; b) repressing transcriptional activity by condensing chromatin; and c) able to bind the region of promoter (for example, zinc finger); and a polypeptide or compound recognizing RNA strand around expression regulatory regions or the cis-acting regions of viral promoter.

In the fusion protein in the present invention, the compounds are compounds recognizing specific nucleic acid sequences joining to proteins by enzymatic or chemical methods. The compounds may be small molecules, nucleic acids analogues or oligo-saccharides.

In the fusion protein of the present invention, the polypeptide or compounds strongly repressing activity of transcription factors such as Sp1 or NF-κB or repressing transcription activity by condensing chromatin, or able to bind transcription regulatory promoter are preferably the polypeptide or compounds selected from the group consisting of: a) POZ-domain proteins; b) HDAC or regions activating transcription inhibition thereof; c) MeCP2 or the analogous MBP-type proteins; d) corepressor proteins selected from the group consisting of polycom family proteins, mSin3A, SMRT and N-CoR; e) DNA binding region polypeptide of Sp1, Sp2, Sp3, Sp4 or NF-_{K}B; and f) proteins(for example; zinc finger) able to bind around HIV promoters.

Polypeptides or compounds recognizing short transcript around expression regulatory regions or the cis-acting regions of viral promoter are preferably Tat protein shown in SEQ ID NO:1 or 2, its derived polypeptide fragments or mutants thereof.

It is more preferable that the fusion protein of the present invention is one or more fusion protein selected from the group consisting of proteins shown in SEQ ID NO:3˜10.

The present invention also provides base sequences of SEQ ID NO:11˜1 8 for coding the polypeptides shown in SEQ ID NO:3˜10 respectively.

A fusion protein in the present invention, the polypeptides shown in SEQ ID NO:1˜2 are Tat protein mutants consisting of 73 and 72 amino acids, respectively, SEQ ID NO:3 is amino acid sequence of MeCP2-TatdMT(73aa) fusion protein, SEQ ID NO:4 is amino acid sequence of HDAC1-TatdMt(73aa) fusion protein, SEQ ID NO:5 is amino acid sequence of POZ-TatdMt(73aa) fusion protein, SEQ ID NO:6 is amino acid sequence of FBI-1-TatdMt(73aa) fusion protein, SEQ ID NO:7 is amino acid sequence of TatdMt(72aa)-MeCP2 fusion protein, SEQ ID NO:8 is amino acid sequence of TatdMt(72aa)-HDAC1 fusion protein, SEQ ID NO:9 is amino acid sequence of TatdMt(72aa)-POZ fusion protein, and SEQ ID NO:10 is amino acid sequence of TatdMt(72aa)-FBI-1 fusion protein.

The present invention also provides base sequences shown in SEQ ID NO:11 for coding the MeCP2-TatdMt(73aa) fusion protein, SEQ ID NO:12 is base sequences coding the HDAC1-TatdMt(73aa), SEQ ID NO:13 is base sequences coding the POZ-TatdMt(73aa), SEQ ID NO:14 is base sequences coding the FBI-1-TatdMt(73aa), SEQ ID NO:15 is base sequences coding the TatdMt(72aa)-MeCP2, SEQ ID NO:16 is base sequences coding the TatdMt(72aa)-HDAc1, SEQ ID NO:17 is base sequences coding TatdMt(72aa)-POZ, and SEQ ID NO:18 is base sequences coding TatdMt(72aa)-FBI-1.

The present invention provides compositions for suppressing proliferation of HIV including a portion or the whole of the fusion proteins.

The present invention provides a portion or the whole of base sequences of one or more recombinant vector selected from the group consisting of pcDNA3.0-TatWt, pcDNA3.0-TatMt, pcDNA3.0-FBI-1, pcDNA3.0-MeCP2-TatWt, pcDNA3.0HDAC1-TatWt, pcDNA3.0FBI-1-TatWt, pcDNA3.0-POZ-TatWt, pcDNA3.0TarWt-MeCP2, pcDNA3.0TatWt-HDAC1, pcDNA3.0TatWt-FBI-1, pcDNA3.0TatWt-POZ, pcDNA3.0-MeCP2-TatdMt, pcDNA3.0HDAC1-TatdMt, pcDNA3.0FBI-1-TatdMt, pcDNA3.0-POZ-TatdMt, pcDNA3.0TatdMt-Mecp2, pcDNA3.0TatdMt-HDAC1, pcDNA3.0TatdMt-FBI-1 and pcDNA3.0TatdMt-POZ comprising genes coding the above-mentioned fusion proteins.

The present invention provides a method for suppressing transcription of viral genome(RNA) by targeting proteins or materials repressing transcription by using protein or material having binding activity to HIV short transcripts or promoter regulatory regions (cis-acting elements).

### BRIEF DESCRIPTION OF THE DRAWINGS

FIG. 1 is a picture illustrating the transcription control of viral genome(RNA) in HIV LTR region. RNA synthesis at the low level is mainly determined by Sp1, NF-κB and TATA box. Defective Sp1-associated GC-Box or TATA box prevents transcription. If transcription is happened once, HIV short strands (short transcripts) exist around LTR promoters. If Tat proteins bind TAR regions, PTEF (positive transcription elongation factor) binds and then CDK9 (cyclin dependent kinase 9) strongly phosphorylates CTD (C Terminal domain) of polymerase II (Pol II). As a result, long viral RNA is formed, thereby rapidly replicating HIV.

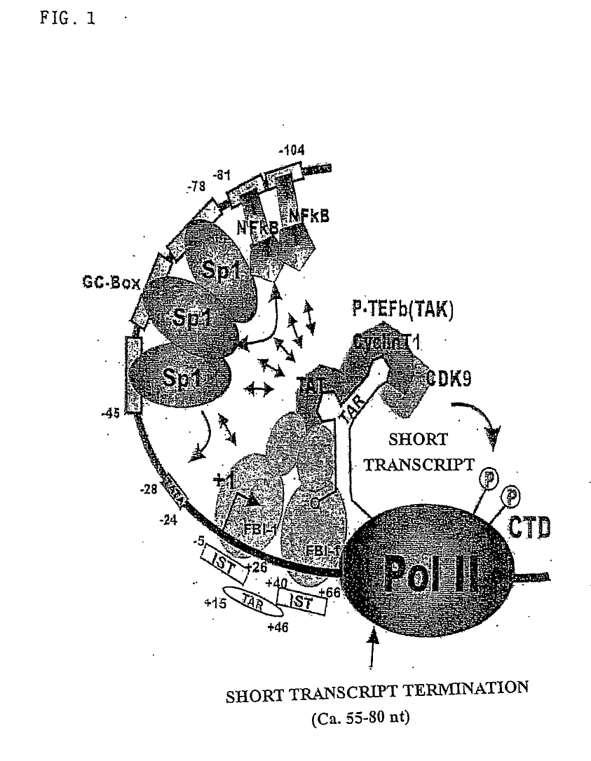

FIG. 2 shows the construct of repressor-TatWt (consisting of wild type 86 amino acid of Tat protein) fusion proteins(a), and (b) shows the analysis result of transient expression in CV-1 cells. The fusion proteins fused with TatWt may not inhibit viral genome expression in HIV-LTR at the level of transcription, but the fusion proteins may be targeted into HIV LTR promoters by TatWt part, thereby enhancing expression by stimulating transcription elongation.

FIG. 3 shows the construct(a) of repressor-TatdMt (mutants consisting of 72 or 73 amino acids wherein 2 amino acid sequences of Tat proteins are mutated), (b) shows the result of transient expression in CV-1 cells, and (c) shows the result of expression in HeLa cells wherein HIV-1 LTR promoters are inserted into genomes like provirus state. The fusion proteins fused with TatdMt strongly inhibit transcription of viral genome in HIV-LTR in the presence of 300 ng of TatWt expression plasmid, thereby reduce the expression by the low level in the absence of Tat. The result reveals that transcription in HIV-LTR like provirus state in HeLa cell strongly inhibit by targeting fusion protein to TAR region by Tat.

FIG. 4 shows function of TatWt and FBI-1, FBI-1-TatdMt, TatdMt in HIV-1 LTR promoters. FBI-1 itself does not show transcription inhibitory function. TatdMt shows only competitive inhibitory function. When the same amount of FBI-1 and TatdMt is provided, about 50% of inhibitory function is shown(b8). But FBI-1-TatdMt shows inhibitory function at 1 ng, and 50% inhibitory function at 3 ng. FBI-1-TatdMt shows the same inhibitory function at 81 ng as that of the absence of TatWt, thereby completely inhibiting transcription by TatWt. However, FBI-1-TatdMt shows a lower level of expression suppression at 243 ng than in the absence of TatWt.

### DETAILED DESCRIPTION OF THE PREFERRED EMBODIMENTS

The present invention will be explained in terms of exemplary embodiments described in detail with reference to the accompanying drawings, which are given only by way of illustration and thus are not limitative of the present invention.

Regulation of synthesizing HIV RNA is complicated, and it requires interaction with cis-acting elements in various viral LTRs, viral transactivators and cellular proteins. This interaction controls the basic level of RNA transcription, and induces expression of high amount of viral genes. Transcription in HIV-LTR promoters is mainly regulated by cellular protein Sp1 transcription factors for recognizing adjacent promoters in the absence of viral protein Tat.

Various signals inducing activation of NF-κB activates transcription by interacting with cis-acting elements existing in upstream of Sp1-associated GC-Box. Other proteins (cofactors) promote transcription and replication in cells having latent HIV provirus by regulating activity of proteins such as Sp1 or NF-κB.

Tat proteins which HIV codes activate transcription by increasing the initiation or elongation of transcription, and they are important for replication. Tat requires TAR, which is a cis-acting RNA element existing in the 5′ end of viral transcripts. Tat also highly promotes production of viral RNA having TAR stem-loop RNA structure in the 5′ end of all HIV transcripts.

As a result of the recent study, Sp1 is repressed by interaction with HDAC and POZ-domain through zinc-finger DNA binding domain, and also by an interaction with MeCP2. The study has demonstrated that a POZ-domain transcription factor referred to as FBI-1 inhibits HIV transcription by interacting with Sp1 and Tat and binding to an IST region (short strand inductive region) of HIV-LTR. These proteins interact with corepressor proteins such as mSin3A, SMRT/N-CoR and HAD for inhibiting transcription by condensing chromatin. Accordingly, if these proteins are targeted in HIV-1 LTR, the activity of transcription by Sp1 and Tat may be regulated by blocking activity of Sp1, condensing chromatin around HIV promoters and targeting polypeptides able to bind HIV promoter region.

Expression plasmid fused transcription inhibitory protein with TatWt is prepared in order to target transcription inhibitory proteins to core promoter regions. Inventors in the present invention make TatWt fusion proteins not inhibit transcription in HIV LTR (promoters) in cell and TatWt fusion proteins function as transcription activation factor of viral RNA by TatWt part of fusion proteins. Function of transcription stimulatory factor of transcription inhibitory proteins fused with TatWt is regarded that TatWt potently functions in transcription elongation step enough to compensate with function of inhibitors inhibiting transcription initiation. However, although fusion proteins consist of two different kinds of proteins, they may be targeted to TAR by TatWt part. As a result, Tat proteins may be used as a targeting means to a core LTR promoter region.

Accordingly, the present invention uses Tat protein mutant (TatdMt:TatK28A&K50A) strongly binding to TAR but lacking an interaction with TAK (or referred to as ‘PTEF’) which is an important cellular factor in transcription activation by Tat.

The longest Tat of HIV consists of 101 amino acids. Because there are many diverse mutants, it is difficult to distinguish which kind of Tats a wild type. The present invention uses Tat proteins wherein Lys-28 and Lys-50 are substituted with alanine, and uses a Tat polypeptide consisting of 72 or 73 amino acids. However, fragments of the Tat polypeptide or other Tat mutants besides the above-mentioned Tat have the similar function as described above.

TatdMt fusion proteins dramatically inhibit transcription of HIV genomes in simian CV-1 cells even when excessive Tat (300 ng of expression plasmid) is expressed.

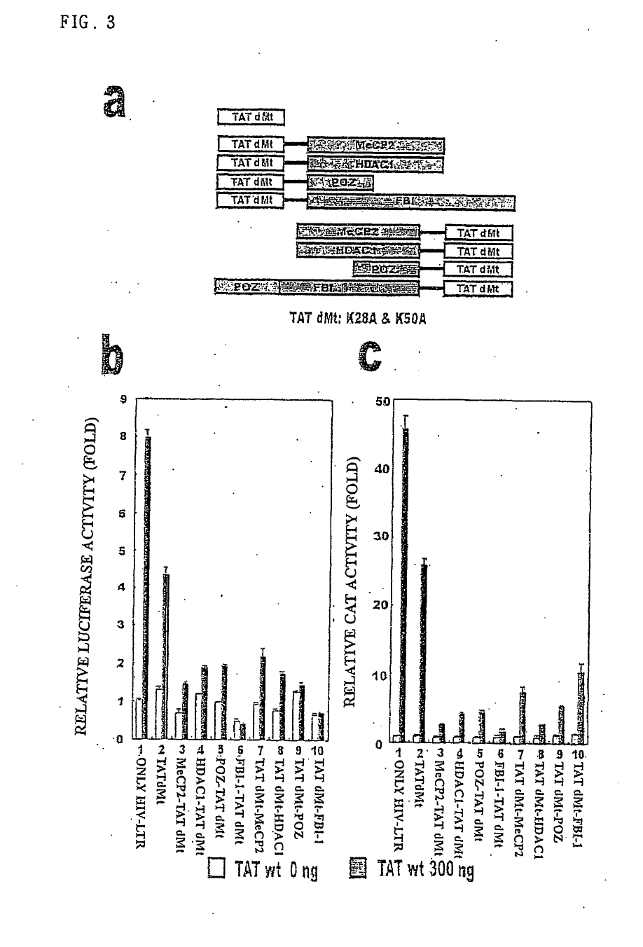

This experimental result is an epoch-making discovery of a protein having a dominant-negative form. Most Tat fusion proteins function regardless of their directions binding to TatdMt. Particularly HDAC-TatdMt, FBI-1-TatdMt fusion proteins strongly inhibit the transcription (see FIG. 3b). The fusion proteins strongly act as inhibitors in HeLa cells wherein HIV-1 LTR-chloramphenicol acetyl transferase gene is inserted into human genomes. Although Tat protein itself activates transcription by 46 times, MeCP2, HDAC, POZ-, or FBI-1-TatdMt fusion proteins strongly inhibit the transcription activation by TatWt even under 300 ng of TatWt over expression plasmids condition. HDAC-TatdMt and FBI-1-TatdMt among the fusion proteins completely inhibit the transcription activation by Tat in HeLa cells too. This result shows that the fusion proteins may repress the transcription of HIV inserted into human genomes as provirus state, in consequence effectively inhibit the proliferation of HIV.

Because the fusion proteins of the present invention repress transcription in HIV LTR promoter and RNA necessary to produce all components for viral replication is not produced, it necessarily follows that the fusion proteins repress expression of viral proteins and replication of HIV.

### Example 1

### Preparation of TatWt Plasmid

HIV-1 LTR CAT fusion plasmid (pUC3R-III CAT) was prepared by cloning about 720 bp of HIV-LTR to pCAT-Basic plasmid (Promega Co.). HIV-1 LTR Luciferase fusion plasmid (pUC3R-III-Luc) was prepared by cloning 720 bp segments of pUC3R-III CAT digested with Xho I HidIII restriction enzymes to pGL3-Basic plasmid (Xho I HidIII, Promega Co.).

The fusion proteins of the present invention fused with Tat (consisting of 86 amino acids), TatdMt (consisting of 73 amino acids), HDAC1, MeCP2, FBI-1, POZ-domain and TatWt or mutant TatdMt (TatK28AK50A) were prepared by amplifying the corresponding gene by a PCR method, and then cloning the genes to a mammalian expression vector pcDNA3.0(Inivtorgen). In order to prepare pcDNA3.0 TatWt(86 amino acids), The genes were amplified by a PCR method using pET-15b-TATWt (86aa, provided by phD. Park Jinseo in Hallym Univ., Korea, HIVHBX2R type) as a template and

5′ primer:
GATCGAATTCATGGAGCCAGTACCTAGACTAGAGCCC,
3′ primer:
GATCTCTAGATCATTCCTTCGGGCCTGTCGGGTCCCCTC.

The reaction conditions were as follows: template denaturation at 94° C. for 3 minutes, 30 cycles of amplification reaction (94° C. 30 sec.; 60° C. 1 min.; 72° C. 30 sec.), and then post-amplification reaction at 72° C. for 3 minutes. After PCR products and expression vector pcDNA3.0 were digested with two restriction enzyme EcoR1 and Xba1, the digested products were ligated using T4 DNA ligase, and then introduced into E. coli DH5a by a transformation method and pcDNA3.0 TatWt(86 amino acids) plasmid was prepared by an alkali lysis method.

Next, in order to prepare pcDNA3.0 TatdMt(73aa), methods similar to the above mentioned methods were used. However, pcDNA3.0TatdMt was prepared by a PCR amplification using HIV Tat gene (Kiernan et al., EMBO J. 18:6106-6118, 1999) as a template and 5′ primer: GAT CGA ATT CAT GGA GCC AGT AAA TCC TAG CCT AG, 3′ Primer: GATCTCTAGATCAGCTTTGATAGAGAAACTTGATG (containing stop codon). In order to prepare HDAC, MeCP2, POZ-, or FBI-1 fusion proteins of X-TatdMT, non-Tat portions were prepared by amplifying the corresponding human cDNA using PCR method and then by cloning the amplified genes to pcDNA3.0 TatdMt. The Reaction conditions were as follows: template denaturation at 95° C. for 3 minutes, 30 cycles of amplification (95° C. 30 sec.; 62° C. 1 min.; 72° C. 7 min.) and post-amplification reaction at 72° C. for 3 minutes, and PCR reaction was carried out using the following PCR primers:

MeCP2
5′ primer:
GATCGGATCCACCATGGTAGCTGGGATGTTAGGGCTCAG
3′ primer:
GATCGAATTCGCTAACTCTCTCGGTCACGGGCGTCCG
HCAC1
5′ primer:
GATCAAGCTTACCATGGCGCAGACGCAGGGCACCCGGAGG
3′ primer:
GATCGAATTCGGCCAACTTGACCTCCTCCTTGACCCCTTTG
POZ-domain
5′ primer:
GATCAAGCTTACCATGGCCGGCGGCGTGGACGGCCCCATC
3′ primer:
GATCGAATTCCTGCCGGTCCAGGAGGTCGGCGCACACG
FBI-1
5′ primer:
GATCAAGCTTACCATGGCCGGCGGCGTGGACGGCCCCATC
3′ primer:
GATCAGATTCGGCGAGTCCGGCTGTGAAGTT.

The amplified products were digested with BamH1-EcoR1 (MeCP2) or HindIII-EcoR1(HDAC1, POZ, FBI-1), and then cloned to pcDNA3.0 TatdMt/BamH1-EcoR1 or pcDNA3.0 TatdMt/HindIII-EcoR1.

In order to prepare HDAC, MeCP2, POZ-, FBI-1 fusion proteins of pcDNA3.0 TatdMt-X, methods similar to the above mentioned methods were used. However, pcDNA3.0 TatdMt(73aa) was prepared by PCR amplification using HIV Tat gene (Kiernan et al., EMBO J. 18: 6106-6118, 1999) as a template and

5′ primer: GATCGGATCCACCATGGACGGAGTAAATCCTAGCCTAG
3′ primer: GATCGAATTCGGGCTTTGATAGAGAAACTTGATG.

Non-Tat portions of pcDNA3.0 TatdMt-x family were prepared by amplifying the corresponding human cDNA in the same condition the above mentioned and by digesting the amplified products with EcoR1-Xba1 and by cloning the digested products to pcDNA3.0 TatdMt/EcoR1-Xba1. Primer sets used in amplification reaction were as follows:

MeCP2
5′ primer: GATCGAATTCATGGTAGCTGGGATGTTAGGGCTCA
3′ primer: GATCTCTAGATCAGCTAACTCTCTCGGTCACGGGC
HDAC1
5′ primer: GATCGAATTCATGGCGCAGACGCAGGGCACCCGGA
3′ primer: GATCTCTAGATCAGGCCAACTTGACCTCCTCCTTG
POZ-domain
5′ primer: GATCGAATTCATGGCCGGCGGCGGCGTGGACGGCC
3′ primer: GATCTCTAGATCACTGCCGGTCCAGGAGGTCGGCG
FBI-1
5′ primer: GATCGAATTCATGGCCGGCGGCGGCGTGGACGGCC
3′ primer: GATCTCTAGATCAGGCGAGTCCGGCTGTGAAGTT.

### Example 2

### Transient Expression Assay

CV-1 cells were cultured in a DMEM culture medium with 10% FBS. When the cells grew enough to occupy 50-60% of bottom area in culture vessel, the mixtures consisting of 0.6 μg of pHIV-LTR-Luciferase plasmid, pCMV-β galactosidase plasmid, TatWt, and a mammalian expression pcDNA3.0 plasmid selected from the group consisting of HDAC1-TatdMt, MeCP2-TatdMt, FBI-1-TatdMt, POZ-domian-TatdMt, TatdMt-HDAC1, TatdMt-MeCP2, TatdMt-FBI-1, and TatdMt-POZ-domain were introduced into cell using a lipopectamin plus reagent(Gibco-BRL).

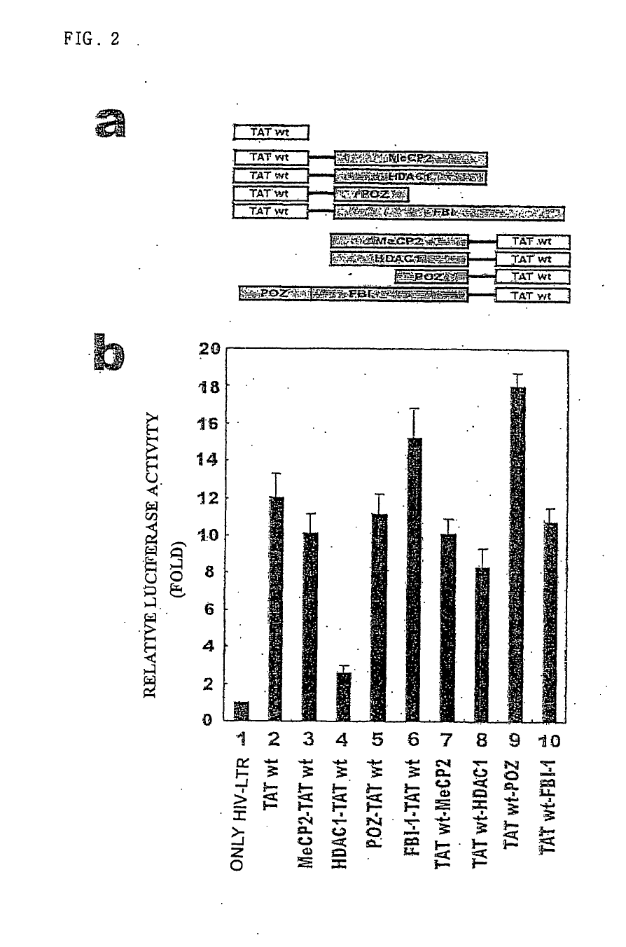

In order to perform an experiment for repressing genome expressions in HeLa cells which HIV-LTR-CAT was inserted into genome, the mixtures consisting of 300 ng of Tat expression plasmid and 300 ng of various fusion proteins expression plasmid of the present invention were introduced into cells were cultured in DMEM culture medium with 10% FBS using the lipopectamin plus reagent (Gibco-BRL). The cells were cultured for 24 hours, and the expression of reporter gene was analyzed. The efficiency for introducing plasmid into cell and the deviation for recovering cell extracts were standardized using activity of simultaneously introduced and expressed β-galactosidase. The results of the present invention are shown in FIGS. 2˜4.

In FIG. 2, the fusion proteins fused with TatWt may not inhibit genome expression in HIV-LTR at transcriptional level, but the fusion proteins may be targeted into HIV LTR promoter region by TatWt part, thereby enhancing expression by promoting transcription elongation.

In FIG. 3, the fusion proteins fused with TatdMt (mutants consisting of 72 or 73 amino acids wherein 2 amino acid sequences of Tat proteins are mutated) potently inhibit genome transcription in HIV-LTR in the presence of 300 ng of TatWt expression plasmid, thereby reduce the expression by the low level in the absence of Tat. The result reveals that transcription in HIV-LTR like provirus state in HeLa cell strongly inhibit by targeting fusion protein to TAR region by Tat.

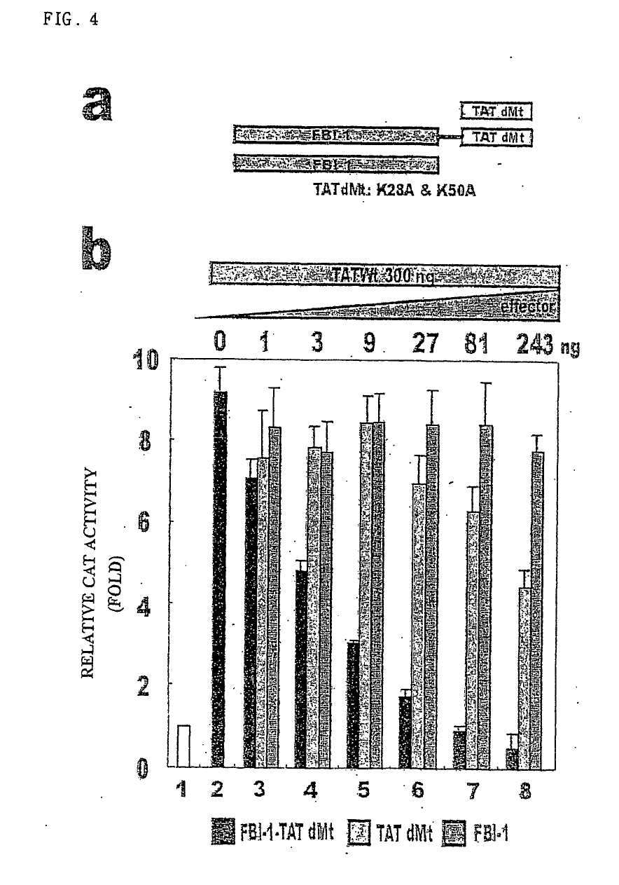

In FIG. 4, FBI-1 itself does not show transcription inhibitory function. TatdMt shows only competitive inhibition that competitively binds to the same site (TAR). When the same amount of FBI-1 and TatdMt is provided, about 50% of inhibitory function is shown(b8). But FBI-1-TatdMt shows inhibitory function at 1 ng, and 50% inhibitory function at 3 ng. FBI-1-TatdMt shows the same inhibitory function at 81 ng as that of the absence of TatWt, thereby completely inhibiting transcription by TatWt. However, FBI-1-TatdMt shows a lower level of expression suppression at 243 ng than in the absence of TatWt.

Within cell nucleus, host cellular proteins such as Sp1, NF-κB act upon HIV transcription regulatory regions. As a result, viral RNA of short RNA strand (short transcript) is produced and it stays around the regions (representing RNA strands binding to polymerase II). If viral protein such as Tat binds to TAR region, RNA synthesis is highly promoted. As a result, a large amount of viral genome (RNA) is produced, and protein components necessary for viral proliferation are produced by using the resulting viral genome(RNA). Accordingly, the most effective method to inhibit viral proliferation is to regulate function of Sp1, NF-ₖB, Tat.

The present invention provides the method to potently inhibit production of viral RNA (transcription process) in viral LTR, when protein for recognizing HIV short transcript regions (e.g. TAR) is targeted to HIV transcription regulatory promoter region, by fusing the protein with the protein selected from the group consisting of proteins repressing transcription factor like Sp1, NF-κB; corepressor or proteins interacting with corepressor; HDAC, or proteins interacting with HDAC repressing transcriptional activity by strongly condensing chromatin; and proteins such as zinc finger having binding activity to viral promoter region.

The present invention is the first study showing the method of potently inhibiting transcription activity in HIV LTR by targeting transcription inhibitory protein groups to HIV-LTR. The present invention also basically inhibits production of components (genomes, proteins) necessary for viral proliferation by repressing expression of viral RNA genome from DNA type viral LTR (promoter) existing as proviral state in host cellular genome. Accordingly, the present invention may basically inhibit the proliferation of virus and production of resistant virus. Particularly, HDAC-TatdMt and FBI-1-TatdMt fusion proteins completely block the process of producing viral genome (RNA).

Therefore, the present invention to overcome the problems of the conventional drugs as AIDS therapeutic agents is the effective protein or gene therapeutic agent to treat AIDS.

What is claimed is:

1. A fusion protein of repressing HIV transcription, comprising:
a transcription inhibitory polypeptide or compound thereof selected from the group consisting of polypeptide strongly repressing the activity of Sp1 or NF-^{K}B transcription factor, polypeptide repressing transcription activity by condensing chromatin, and a polypeptide having binding activity to promoter; and
a polypeptide or compound thereof recognizing RNA strand around expression control regions or viral LTR promoter cis-acting element.

2. The fusion protein according to claim 1, wherein the polypeptide or the compound thereof strongly repressing transcription activity of Sp1 or NF-_{K}B transcription factor or the polypeptide of the compound thereof repressing transcription activity by condensing chromatin is a polypeptide selected from the group consisting of: a) POZ-domain family proteins; b) HDAC or region with transcription inhibitory activity thereof; c) MeCP2 or MBP-family proteins; d) corepressor proteins selected from the group consisting of polycom family proteins, mSin3A, SMRT, B-CoR and N-CoR; e) polypeptides of DNA binding region of Sp1, Sp2, Sp3, Sp4 or NF-_{K}B; and f) proteins with binding activity to bind to HIV promoter region.

3. The fusion protein according to claim 1, wherein the polypeptide or compound thereof recognizing RNA strand around expression control regions or viral LTR promoter cis-acting element polypeptide or the compound thereof is a Tat protein SEQ ID NO:1 or 2 or mutants thereof.

4. The fusion protein as claimed in any one of the preceding claims, wherein the fission protein is selected from the group consisting of proteins shown in SEQ ID NO:3˜10.

5. A nucleic acid comprising base sequence of SEQ ID NO:11˜18, coding the polypeptides shown in SEQ ID NO:31˜10.

6. One or more recombinant vectors comprising genes for coding the fusion proteins according to claim 5, wherein the recombinant vector is vector selected from the group consisting of pcDNA3.0-TatWt, pcDNA3.0-TatMt, pcDNA3.0-FBI-1, pcDNA3.0-MeCP2-TatWt, pcDNA3.0HDAC1-TatWt, pcDNA3.0FBI-1-TatWt, pcDNA3.0-POZ-TatWt, pcDNA3.0TarWt-MeCP2, pcDNA3.0TatWt-HDAC1, pcDNA3.0TatWt-FBI-1, pcDNA3.0TatWt-POZ, pcDNA3.0-MeCP2-TatdMt, pcDNA3.0HDAC1-TatdMt, pcDNA3.0FBI-1-TatdMt, pcDNA3.0-POZ-TatdMt, pcDNA3.0TatdMt-Mecp2, pcDNA3.0TatdMt-HDAC1, pcDNA3.0TatdMt-FBI-1 and pcDNA3.0TatdMt-POZ.

7. A composition of repressing HIV transcription according to claim 1 or 4, comprising a portion or the whole of the fusion protein.

8. A method for repressing proliferation of HIV by inhibiting genome transcription in the viral LTR and the resulting viral replication by using the fusion proteins.


SEQUENCE LISTING (40 sequences)

SEQ ID NO: 1 - protein, 73 aa, Artificial Sequence
  73 amino acid Tat mutant
MEPVNPSLEPWKHPGSQPKTACTNCYCAKCCFHCQVCFITKALGISYGRAKRRQRRRPPQGSQTHQVSLSKLI

SEQ ID NO: 2 - protein, 72 aa, Artificial Sequence
  72 amino acid Tat mutant
MEPVNPSLEPWKHPGSQPKTACTNCYCAKCCFHCQVCFITKALGISYGRAKRRQRRRPPQGSQTHQVSLSKL

SEQ ID NO: 3 - protein, 561 aa, Artificial Sequence
  MeCP2-Tat dMt fusion protein
MVAGMLGLREEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVSEFMEPVNPSLEPWKHPGSQPKTACTNCYCAKCCFHCQVCFITKALGISYGRAKRRQRRRPPQGSQTHQVSLSKLI

SEQ ID NO: 4 - protein, 557 aa, Artificial Sequence
  HDAC1-TAT dMt fusion protein
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCWTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLAEFMEPVNPSLEPWKHPGSQPKTACTNCYCAKCCFHCQVCFITKALGISYGRAKRRQRRRPPQGSQTHQVSLSKLI

SEQ ID NO: 5 - protein, 205 aa, Artificial Sequence
  POZ-TAT dMt fusion protein
MAGGVDGPIGIPFPDHSSDILSGLNEQRTQGLLCDVVILVEGREFPTHRSVLAACSQYFKKLFTSGAVVDQQNVYEIDFVSAEALTALMDFAYTATLTVSTANVGDILSAARLLEIPAVSHVCADLLDRQEFMEPVNPSLEPWKHPGSQPKTACTNCYCAKCCFHCQVCFITKALGISYGRAKRRQRRRPPQGSQTHQVSLSKLI

SEQ ID NO: 6 - protein, 659 aa, Artificial Sequence
  FBI-1-TAT dMt fusion protein
MAGGVDGPIGIPFPDHSSDILSGLNEQRTQGLLCDVVILVEGREFPTHRSVLAACSQYFKKLFTSGAVVDQQNVYEIDFVSAEALTALMDFAYTATLTVSTANVGDILSAARLLEIPAVSHVCADLLDRQILAADAGADAGQLDLVDQIDQRNLLRAKEYLEFFQSNPMNSLPPAAAAAAASFPWSAFGASDDDLDATKEAVAAAVAAVAAGDCNGLDFYGPGPPAERPPTGDGDEGDSNPGLWPERDEDAPTGGLFPPPVAPPAATQNGHYGRGGEEEAASLSEAAPEPGDSPGFLSGAAEGEDGDGPDVDGLAASTLLQQMMSSVGRAGAAAGDSDEESRADDKGVMDYYLKYFSGAHDGDVYPAWSQKVEKKIRAKAFQKCPICEKVIQGAGKLPRHIRTHTGEKPYECNICKVRFTRQDKLKVHMRKHTGEKPYLCQQCGAAFAHNYDLKNHMRVHTGLRPYQCDSCCKTFVRSDHLHRHLKKDGCNGVPSRRGRKPRVRGGAPDPSPGATATPGAPAQPSSPDARRNGQEKHFKDEDEDEDVASPDGLGRLNVAGAGGGGDSGGGPGAATDGNFTAGLAEFMEPVNPSLEPWKHPGSQPKTACTNCYCAKCCFHCQVCFITKALGISYGRAKRRQRRRPPQGSQTHQVSLSKLI

SEQ ID NO: 7 - protein, 560 aa, Artificial Sequence
  TAT dMt-MeCP2 fusion protein
MEPVNPSLEPWKHPGSQPKTACTNCYCAKCCFHCQVCFITKALGISYGRAKRRQRRRPPQGSQTHQVSLSKPEFMVAGMLGLREEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 8 - protein, 556 aa, Artificial Sequence
  TAT dMt-HDAC1 fusion protein
MEPVNPSLEPWKHPGSQPKTACTNCYCAKCCFHCQVCFITKALGISYGRAKRRQRRRPPQGSQTHQVSLSKPEFMAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCWTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 9 - protein, 204 aa, Artificial Sequence
  TAT dMt-POZ fusion protein
MEPVNPSLEPWKHPGSQPKTACTNCYCAKCCFHCQVCFITKALGISYGRAKRRQRRRPPQGSQTHQVSLSKPEFMAGGVDGPIGIPFPDHSSDILSGLNEQRTQGLLCDVVILVEGREFPTHRSVLAACSQYFKKLFTSGAVVDQQNVYEIDFVSAEALTALMDFAYTATLTVSTANVGDILSAARLLEIPAVSHVCADLLDRQ

SEQ ID NO: 10 - protein, 658 aa, Artificial Sequence
  TAT dMt-FBI-1 fusion protein
MEPVNPSLEPWKHPGSQPKTACTNCYCAKCCFHCQVCFITKALGISYGRAKRRQRRRPPQGSQTHQVSLSKPEFMAGGVDGPIGIPFPDHSSDILSGLNEQRTQGLLCDVVILVEGREFPTHRSVLAACSQYFKKLFTSGAVVDQQNVYEIDFVSAEALTALMDFAYTATLTVSTANVGDILSAARLLEIPAVSHVCADLLDRQILAADAGADAGQLDLVDQIDQRNLLRAKEYLEFFQSNPMNSLPPAAAAAAASFPWSAFGASDDDLDATKEAVAAAVAAVAAGDCNGLDFYGPGPPAERPPTGDGDEGDSNPGLWPERDEDAPTGGLFPPPVAPPAATQNGHYGRGGEEEAASLSEAAPEPGDSPGFLSGAAEGEDGDGPDVDGLAASTLLQQMMSSVGRAGAAAGDSDEESRADDKGVMDYYLKYFSGAHDGDVYPAWSQKVEKKIRAKAFQKCPICEKVIQGAGKLPRHIRTHTGEKPYECNICKVRFTRQDKLKVHMRKHTGEKPYLCQQCGAAFAHNYDLKNHMRVHTGLRPYQCDSCCKTFVRSDHLHRHLKKDGCNGVPSRRGRKPRVRGGAPDPSPGATATPGAPAQPSSPDARRNGQEKHFKDEDEDEDVASPDGLGRLNVAGAGGGGDSGGGPGAATDGNFTAGLA

SEQ ID NO: 11 - DNA, 1686 bp, Artificial Sequence
  base sequence of MeCP2-TAT dMt fusion protein
atggtagctgggatgttagggctcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaagctgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagcgaattcatggagccagtaaatcctagcctagagccctggaagcatccaggaagtcagcctaaaactgcttgtaccaattgctattgtgcaaagtgttgctttcattgccaagtttgtttcataacaaaagccttaggcatctcctatggcagggcaaagcggagacagcgacgaagacctcctcaaggcagtcagactcatcaagtttctctatcaaagctgatctag

SEQ ID NO: 12 - DNA, 1674 bp, Artificial Sequence
  base sequence of HDAC1-TAT dMt fusion protein
atggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggccgaattcatggagccagtaaatcctagcctagagccctggaagcatccaggaagtcagcctaaaactgcttgtaccaattgctattgtgcaaagtgttgctttcattgccaagtttgtttcataacaaaagccttaggcatctcctatggcagggcaaagcggagacagcgacgaagacctcctcaaggcagtcagactcatcaagtttctctatcaaagctgatctag

SEQ ID NO: 13 - DNA, 618 bp, Artificial Sequence
  base sequence of POZ-TAT dMt fusion protein
atggccggcggcgtggacggccccatcgggatcccgttccccgaccacagcagcgacatcctgagtgggctgaacgagcagcggacgcagggcctgctgtgcgacgtggtgatcctggtggagggccgcgagttccccacgcaccgctcggtgctggccgcctgcagccagtacttcaagaagctgttcacgtcgggcgccgtggtggaccagcagaacgtgtacgagatcgacttcgtcagcgccgaggcgctcaccgcgctcatggacttcgcctacacggccacgctcaccgtcagcacagccaacgtgggtgacatcctcagcgccgcccgcctgctggagatccccgccgtgagccacgtgtgcgccgacctcctggaccggcaggaattcatggagccagtaaatcctagcctagagccctggaagcatccaggaagtcagcctaaaactgcttgtaccaattgctattgtgcaaagtgttgctttcattgccaagtttgtttcataacaaaagccttaggcatctcctatggcagggcaaagcggagacagcgacgaagacctcctcaaggcagtcagactcatcaagtttctctatcaaagctgatctag

SEQ ID NO: 14 - DNA, 1980 bp, Artificial Sequence
  base sequence of FBI-1-TAT dMt fusion protein
atggccggcggcgtggacggccccatcgggatcccgttccccgaccacagcagcgacatcctgagtgggctgaacgagcagcggacgcagggcctgctgtgcgacgtggtgatcctggtggagggccgcgagttccccacgcaccgctcggtgctggccgcctgcagccagtacttcaagaagctgttcacgtcgggcgccgtggtggaccagcagaacgtgtacgagatcgacttcgtcagcgccgaggcgctcaccgcgctcatggacttcgcctacacggccacgctcaccgtcagcacagccaacgtgggtgacatcctcagcgccgcccgcctgctggagatccccgccgtgagccacgtgtgcgccgacctcctggaccggcagatcctggcggccgacgcgggcgccgacgccgggcagctggaccttgtagatcaaattgatcagcgcaacctcctccgcgccaaggagtacctcgagttcttccagagcaaccccatgaacagcctgccccccgcggccgccgccgccgctgccagcttcccgtggtccgcctttggggcgtccgatgatgacctggatgccaccaaggaggccgtggccgccgctgtggccgccgtggccgcgggcgactgcaacggcttagacttctatgggccgggccccccggccgagcggcccccgacgggggacggggacgagggcgacagcaacccgggtctgtggccagagcgggatgaggacgcccccaccgggggtctctttccgccgccggtggccccgccggccgccacgcagaacggccactacggccgcggcggagaggaggaggccgcctcgctgtcggaggcggcccccgagccgggcgactctccgggcttcctgtcgggagcggccgagggcgaggacggggacgggcccgacgtggacgggctggcggccagcacgctgctgcagcagatgatgtcatcggtgggccgggcgggggccgcggcgggggacagcgacgaggagtcgcgggccgacgacaagggcgtcatggactactacctgaagtacttcagcggcgcccacgacggcgacgtctacccggcctggtcgcagaaggtggagaagaagatccgagccaaggccttccagaagtgccccatctgcgagaaggtcatccagggcgccggcaagctgccgcgacacatccgcacccacacgggcgagaagccctacgagtgcaacatctgcaaggtccgcttcaccaggcaggacaagctgaaggtgcacatgcggaagcacacgggcgagaagccgtacctgtgccagcagtgcggcgccgcctttgcccacaactacgacctgaagaaccacatgcgcgtgcacacgggcctgcgcccctaccagtgcgacagctgctgcaagaccttcgtccgctccgaccacctgcacagacacctcaagaaagacggctgcaacggcgtcccctcgcgccgcggccgcaagccccgcgtccggggcggggcgcccgaccccagcccgggggccaccgcgacccccggcgcccccgcccagcccagctcccccgacgcccggcgcaacggccaggagaagcactttaaggacgaggacgaggacgaggacgtggccagccccgacggcttgggccggttgaatgtagcgggcgccggtggaggaggtgacagcggaggtggccccggggccgccaccgacggtaacttcacagccggactcgccgaattcatggagccagtaaatcctagcctagagccctggaagcatccaggaagtcagcctaaaactgcttgtaccaattgctattgtgcaaagtgttgctttcattgccaagtttgtttcataacaaaagccttaggcatctcctatggcagggcaaagcggagacagcgacgaagacctcctcaaggcagtcagactcatcaagtttctctatcaaagctgatctag

SEQ ID NO: 15 - DNA, 1683 bp, Artificial Sequence
  base sequence of TAT dMt-MeCP2 fusion protein
atggagccagtaaatcctagcctagagccctggaagcatccaggaagtcagcctaaaactgcttgtaccaattgctattgtgcaaagtgttgctttcattgccaagtttgtttcataacaaaagccttaggcatctcctatggcagggcaaagcggagacagcgacgaagacctcctcaaggcagtcagactcatcaagtttctctatcaaagcccgaattcatggtagctgggatgttagggctcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaagctgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctga

SEQ ID NO: 16 - DNA, 1671 bp, Artificial Sequence
  base sequence of TAT dMt-HDAC1 fusion protein
atggagccagtaaatcctagcctagagccctggaagcatccaggaagtcagcctaaaactgcttgtaccaattgctattgtgcaaagtgttgctttcattgccaagtttgtttcataacaaaagccttaggcatctcctatggcagggcaaagcggagacagcgacgaagacctcctcaaggcagtcagactcatcaagtttctctatcaaagcccgaattcatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctga

SEQ ID NO: 17 - DNA, 612 bp, Artificial Sequence
  base sequence of TAT dMt-POZ fusion protein
atggagccagtaaatcctagcctagagccctggaagcatccaggaagtcagcctaaaactgcttgtaccaattgctattgtgcaaagtgttgctttcattgccaagtttgtttcataacaaaagccttaggcatctcctatggcagggcaaagcggagacagcgacgaagacctcctcaaggcagtcagactcatcaagtttctctatcaaagcccgaattcatggccggcggcgtggacggccccatcgggatcccgttccccgaccacagcagcgacatcctgagtgggctgaacgagcagcggacgcagggcctgctgtgcgacgtggtgatcctggtggagggccgcgagttccccacgcaccgctcggtgctggccgcctgcagccagtacttcaagaagctgttcacgtcgggcgccgtggtggaccagcagaacgtgtacgagatcgacttcgtcagcgccgaggcgctcaccgcgctcatggacttcgcctacacggccacgctcaccgtcagcacagccaacgtgggtgacatcctcagcgccgcccgcctgctggagatccccgccgtgagccacgtgtgcgccgacctcctggaccggcag

SEQ ID NO: 18 - DNA, 1977 bp, Artificial Sequence
  base sequence of TAT dMt-FBI-1 fusion protein
atggagccagtaaatcctagcctagagccctggaagcatccaggaagtcagcctaaaactgcttgtaccaattgctattgtgcaaagtgttgctttcattgccaagtttgtttcataacaaaagccttaggcatctcctatggcagggcaaagcggagacagcgacgaagacctcctcaaggcagtcagactcatcaagtttctctatcaaagcccgaattcatggccggcggcgtggacggccccatcgggatcccgttccccgaccacagcagcgacatcctgagtgggctgaacgagcagcggacgcagggcctgctgtgcgacgtggtgatcctggtggagggccgcgagttccccacgcaccgctcggtgctggccgcctgcagccagtacttcaagaagctgttcacgtcgggcgccgtggtggaccagcagaacgtgtacgagatcgacttcgtcagcgccgaggcgctcaccgcgctcatggacttcgcctacacggccacgctcaccgtcagcacagccaacgtgggtgacatcctcagcgccgcccgcctgctggagatccccgccgtgagccacgtgtgcgccgacctcctggaccggcagatcctggcggccgacgcgggcgccgacgccgggcagctggaccttgtagatcaaattgatcagcgcaacctcctccgcgccaaggagtacctcgagttcttccagagcaaccccatgaacagcctgccccccgcggccgccgccgccgctgccagcttcccgtggtccgcctttggggcgtccgatgatgacctggatgccaccaaggaggccgtggccgccgctgtggccgccgtggccgcgggcgactgcaacggcttagacttctatgggccgggccccccggccgagcggcccccgacgggggacggggacgagggcgacagcaacccgggtctgtggccagagcgggatgaggacgcccccaccgggggtctctttccgccgccggtggccccgccggccgccacgcagaacggccactacggccgcggcggagaggaggaggccgcctcgctgtcggaggcggcccccgagccgggcgactctccgggcttcctgtcgggagcggccgagggcgaggacggggacgggcccgacgtggacgggctggcggccagcacgctgctgcagcagatgatgtcatcggtgggccgggcgggggccgcggcgggggacagcgacgaggagtcgcgggccgacgacaagggcgtcatggactactacctgaagtacttcagcggcgcccacgacggcgacgtctacccggcctggtcgcagaaggtggagaagaagatccgagccaaggccttccagaagtgccccatctgcgagaaggtcatccagggcgccggcaagctgccgcgacacatccgcacccacacgggcgagaagccctacgagtgcaacatctgcaaggtccgcttcaccaggcaggacaagctgaaggtgcacatgcggaagcacacgggcgagaagccgtacctgtgccagcagtgcggcgccgcctttgcccacaactacgacctgaagaaccacatgcgcgtgcacacgggcctgcgcccctaccagtgcgacagctgctgcaagaccttcgtccgctccgaccacctgcacagacacctcaagaaagacggctgcaacggcgtcccctcgcgccgcggccgcaagccccgcgtccggggcggggcgcccgaccccagcccgggggccaccgcgacccccggcgcccccgcccagcccagctcccccgacgcccggcgcaacggccaggagaagcactttaaggacgaggacgaggacgaggacgtggccagccccgacggcttgggccggttgaatgtagcgggcgccggtggaggaggtgacagcggaggtggccccggggccgccaccgacggtaacttcacagccggactcgcctaa

SEQ ID NO: 19 - DNA, 37 bp, Artificial Sequence
  Primer
gatcgaattcatggagccagtacctagactagagccc

SEQ ID NO: 20 - DNA, 39 bp, Artificial Sequence
  Primer
gatctctagatcattccttcgggcctgtcgggtcccctc

SEQ ID NO: 21 - DNA, 35 bp, Artificial Sequence
  Primer
gatcgaattcatggagccagtaaatcctagcctag

SEQ ID NO: 22 - DNA, 35 bp, Artificial Sequence
  Primer
gatctctagatcagctttgatagagaaacttgatg

SEQ ID NO: 23 - DNA, 39 bp, Artificial Sequence
  Primer
gatcggatccaccatggtagctgggatgttagggctcag

SEQ ID NO: 24 - DNA, 37 bp, Artificial Sequence
  Primer
gatcgaattcgctaactctctcggtcacgggcgtccg

SEQ ID NO: 25 - DNA, 40 bp, Artificial Sequence
  Primer
gatcaagcttaccatggcgcagacgcagggcacccggagg

SEQ ID NO: 26 - DNA, 41 bp, Artificial Sequence
  Primer
gatcgaattcggccaacttgacctcctccttgacccctttg

SEQ ID NO: 27 - DNA, 40 bp, Artificial Sequence
  Primer
gatcaagcttaccatggccggcggcgtggacggccccatc

SEQ ID NO: 28 - DNA, 38 bp, Artificial Sequence
  Primer
gatcgaattcctgccggtccaggaggtcggcgcacacg

SEQ ID NO: 29 - DNA, 40 bp, Artificial Sequence; identical to SEQ ID NO: 27
  Primer

SEQ ID NO: 30 - DNA, 31 bp, Artificial Sequence
  Primer
gatcagattcggcgagtccggctgtgaagtt

SEQ ID NO: 31 - DNA, 38 bp, Artificial Sequence
  Primer
gatcggatccaccatggacggagtaaatcctagcctag

SEQ ID NO: 32 - DNA, 34 bp, Artificial Sequence
  Primer
gatcgaattcgggctttgatagagaaacttgatg

SEQ ID NO: 33 - DNA, 35 bp, Artificial Sequence
  Primer
gatcgaattcatggtagctgggatgttagggctca

SEQ ID NO: 34 - DNA, 35 bp, Artificial Sequence
  Primer
gatctctagatcagctaactctctcggtcacgggc

SEQ ID NO: 35 - DNA, 35 bp, Artificial Sequence
  Primer
gatcgaattcatggcgcagacgcagggcacccgga

SEQ ID NO: 36 - DNA, 35 bp, Artificial Sequence
  Primer
gatctctagatcaggccaacttgacctcctccttg

SEQ ID NO: 37 - DNA, 35 bp, Artificial Sequence
  Primer
gatcgaattcatggccggcggcggcgtggacggcc

SEQ ID NO: 38 - DNA, 35 bp, Artificial Sequence
  Primer
gatctctagatcactgccggtccaggaggtcggcg

SEQ ID NO: 39 - DNA, 35 bp, Artificial Sequence; identical to SEQ ID NO: 37
  Primer

SEQ ID NO: 40 - DNA, 34 bp, Artificial Sequence
  Primer
gatctctagatcaggcgagtccggctgtgaagtt